(19) United States
(12) Patent Application Publication
(10) Pub. No.: US 2022/0409048 A1
(43) Pub. Date: Dec. 29, 2022

(54) SYSTEMS, METHODS, AND APPARATUSES FOR DETECTING AND QUANTIFYING BIOLOGICAL FLUIDS

(71) Applicant: Bioserenity, Paris (FR)

(72) Inventors: Charles Beye, Paris (FR); Pierre-Yves Frouin, Paris (FR)

(73) Assignee: Bioserenity, Paris (FR)

(21) Appl. No.: 17/355,924

(22) Filed: Jun. 23, 2021

(51) Int. Cl. A61B 5/00; A61B 5/145; A61B 5/1455; A61B 5/053

(52) U.S. Cl. CPC A61B 5/0002; A61B 5/14507; A61B 5/1455; A61B 5/053; A61B 5/6804; A61B 5/4836; A61B 2562/0271

(57) ABSTRACT

Systems, apparatuses, and methods for detecting and quantifying a fluid are described herein. In some examples, a sensor apparatus is used to absorb discharged fluid (or other fluid present). The sensor apparatus includes one or more sensors designed to provide a capacitance measurement using electrodes in the sensors and one or more fluid properties sensors that determine at least one fluid property of an absorbed fluid. The electrodes are designed using mirror image axes of deformation to minimize the effect of deformation caused by the wearer of the sensor while still allowing some degree of flexibility for comfort. The person wearing one or more of the sensors may be monitored and cared for remotely using a communication system between the local device and a remote device used by a caregiver or medical practitioner.

### BACKGROUND

The detection of the presence of fluids, including biological fluids, can be helpful when treating various human issues. For example, a person wearing a gauze may want to know whether or not bleeding underneath the gauze has recommenced. In another example relating to plastic surgery, patients in post-surgery often wear tightly fitting gowns that help facilitate healing and the flow of fluid from openings made during surgery. It may be desirable to monitor for the flow of fluid from these openings to ensure that the amount flowing is correct or within expected parameters. For example, it may be desirable to detect the presence of urine for the detection of incontinence issues.

It is with these and other concerns that various examples of the presently disclosed subject matter are described herein.

### BRIEF DESCRIPTION OF THE DRAWINGS

The detailed description is set forth with reference to the accompanying figures. The use of the same reference numbers in different figures indicates similar or identical items or features.

FIG. 1 is a top down illustration of a sensor that may be used to detect fluid, in accordance with some example of the present disclosure.

FIG. 2 are illustrations showing other examples of shapes of electrodes that have mirror images of sides that allow for countering the effect of deformation, in accordance with some examples of the present disclosure.

FIG. 3 is side, cutaway view of a sensor, in accordance with some examples of the present disclosure.

FIG. 4 illustrates a sensor apparatus, in accordance with some examples of the present disclosure.

FIG. 5 is a front illustration of the placement of a sensor apparatus for use in wound healing, in accordance with some examples of the present disclosure.

FIG. 6 is an illustration of a patient treatment system, in accordance with some examples of the present disclosure.

FIG. 7 is an illustration of a remote user interface as rendered on a mobile device, in accordance with some examples of the present disclosure.

FIG. 8 is an example process for fluid detection and quantification, in accordance with some examples of the present disclosure.

FIG. 9 depicts a component level view of a local monitoring system for use with the systems and methods described herein, in accordance with some examples of the present disclosure.

FIG. 10 is a top down view of a sensor apparatus having a capacitance and fluid properties sensor, in accordance with some examples of the present disclosure.

FIG. 11 is an illustration of a local patient treatment system, in accordance with some examples of the present disclosure.

### DETAILED DESCRIPTION

Examples of the present disclosure comprises systems, apparatuses, and methods for detecting and quantifying a fluid. To detect a fluid, various examples of the presently disclosed subject matter include a fluid sensor. The sensor includes electrodes that are energized using various forms of current/voltage at various frequencies (depending on the particular application). The capacitance between the electrodes is measured, providing an output of the presence of a fluid, and in some examples, the type, an amount, and/or a flowrate of the fluid. The electrodes are sheathed within an impermeable, or only slightly permeable, shield to reduce the probability that the fluid, or other fluids, enter the interstitial space between the electrodes, which potentially may cause shorts or erroneous readings. In some examples, the electrode is geometrically designed to minimize effects of movement of the wearer of the fluidic sensor.

In some examples, the electrode includes a first lead and a second lead, each of the leads connected to a capacitance measurement device. The capacitance measurement device energizes the electrodes. Fluid proximate to the electrode modifies the capacitance of the electrode. The changing capacitance of the electrode is determined by a change of a resonance frequency of a resister/capacitor/inductor (RLC) circuit, which the electrode is part of. The presence of a liquid proximate to the outer surface of the protective shield changes the capacitance of the electrodes, providing an indication of the presence of a liquid. The electrode within the shield may be placed in a garment, brief, pad, bandage, or other material.

Examples of conditions or pathologies where various examples of the presently disclosed subject matter may be used include, but are not limited to, spontaneous cerebrospinal fluid leak, amniotic fluid leakage, lymphorrhagia, exuding wounds (lymph, blood, purulent wound, and the like), diarrhea, vomiting, excessive tear secretion for a person in a coma, excessive secretion of saliva for a person in a coma, various types of urinary diseases and conditions, and excessive sweating at night a sign of low blood sugar. In the context of using a leakage detector for testing for the presence of blood, examples include, but are not limited to, fibroids (such as non-cancerous growths that develop in or around the womb and can cause heavy or painful periods), endometriosis where the tissue that lines the womb (endometrium) is found outside the womb, such as in the ovaries and fallopian tubes, adenomyosis when tissue from the womb lining becomes embedded in the wall of the womb, pelvic inflammatory disease (PID), endometrial polyps, cancer of the womb (in which a common symptom is abnormal bleeding, especially after the menopause), polycystic ovary syndrome (PCOS), blood clotting disorders such as Von Willebrand disease, an underactive thyroid gland (hypothyroidism), and diabetes. It should be understood that the aforementioned conditions and pathologies are merely examples and are not intended as limitations of the scope of the presently disclosed subject matter.

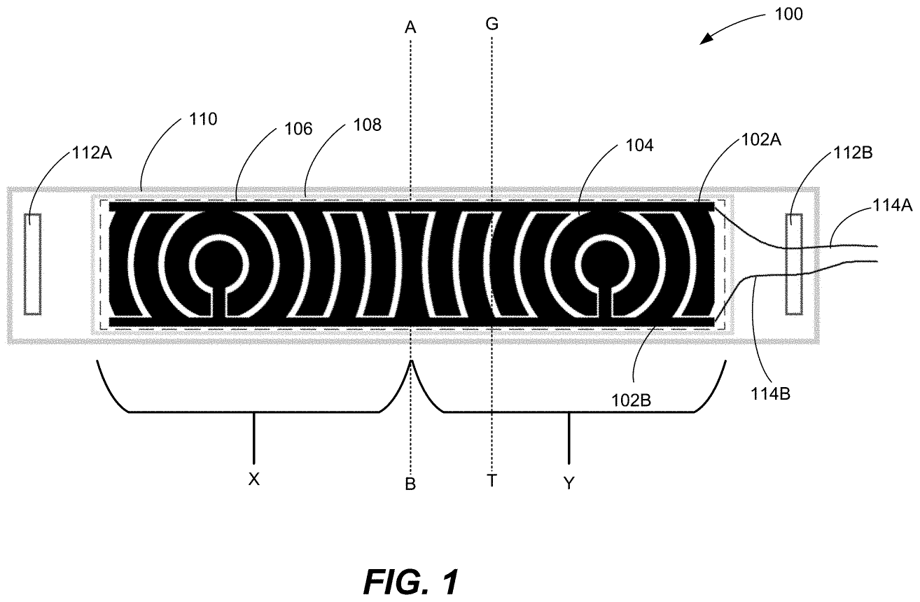

FIG. 1 is a top down illustration of a sensor 100 that may be used to detect fluid, in accordance with some example of the present disclosure. The sensor 100 includes an electrode 102A and an electrode 102B. The electrodes 102A and 102B are separated with an interstitial space 104 that reduces or eliminates the potential of physical contact between the electrodes 102A and 102B, electrically creating a capacitor. During use when placed in a garment, brief, pad, bandage, or other material worn by a person, the sensor 100 may be moved, twisted, bent, folded, and be placed under other force loads that deforms the sensor 100. An issue when a sensor is deformed is that the capacitance of the sensor, when measured, may be significantly altered. The reason for this is that capacitance can be affected by the physical dimensions and placement of the electrodes with respect to each other (thus affecting the dielectric properties). When the electrodes move with respect to each other, the dielectric properties, or dielectric distribution, may be changed, thus changing the capacitance of a capacitor.

When used in electrical or electronic circuits where the capacitor is a single, solid unit that stabilizes the electrodes within, the movement of the capacitor has minimal to no effect on the on the capacitance change with respect to surrounding dielectric distribution. However, having a solid piece metal or other material encapsulating a capacitor may make the use of the capacitor uncomfortable or noticeable when used in a garment, brief, gauze or other material worn by a person. Having a flexible, yet accurate, capacitor can be useful in the context of the sensor 100 when worn by a moving human. However, as noted above, the movement of the electrodes 102A and 102B with respect to each other (and other sensors not illustrated) can affect the capacitance measured.

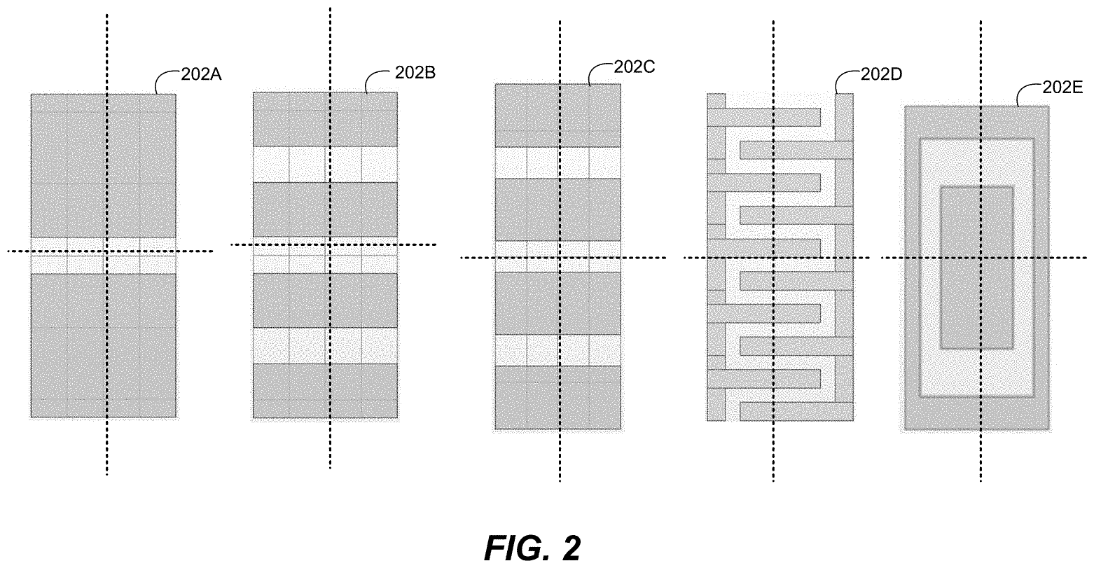

To alleviate issues relating to the flexing, twisting, or other deformations of the sensor 100 when a user moves, the electrodes 102A and 102B are shaped to provide a countering effect to the deformation. Illustrated in FIG. 1 is deformation axis AB. The deformation axis AB delineates “mirror image” sides X and Y. The use of mirror image sides X and Y, which each or effectively one half of the electrodes 102A and 102B, can help alleviate issues relating to deformation by providing a counter effect. For example, if the sensor 100 is deformed along line GT, the effect of the deformation may be minimized by a non-deformation, or opposite directional deformation of the similar, but opposite in geometry, features on side X. Thus, while side Y may be twisted or deformed in one direction, the side X may be partially twisted or deformed in an opposite direction. FIG. 2 are illustrations showing other examples of shapes of electrodes 202A-202F that have mirror images of sides that allow for countering the effect of deformation, in accordance with some examples of the present disclosure. The axes of deformation are illustrated as dashed lines in each. It should be noted that the presently disclosed subject matter is not limited to the particular shapes of the electrodes, as any electrode with mirror images may be used and are considered to be within the scope of the presently disclosed subject matter.

Returning to FIG. 1, the electrodes 102A and 102B may be encapsulated, partially or wholly, within shield 106. The shield 106 comprises an internal volume that is sized and shaped to receive the electrodes 102A and 102B. In some examples, the shield 106 is a polymeric/plastic material or natural fiber (or combinations thereof) based material that provides at least a modicum of protection against impacts onto the electrodes 102A and 102B. In other examples, the shield 106 may also be a polymeric/plastic material or natural fiber (or combinations thereof) that provides a degree of rigidity to the electrodes 102A and 102B to minimize deformation of the electrodes 102A and 102B, while allowing some degree of flexibility of the sensor 100. In still further examples, the shield 106 comprises a metallic or semi-metallic material that acts as an electromagnetic shield. The shield 106 may further be encapsulated, partially or wholly, within insulator 108. The insulator 108 may be partially or fully impermeable to liquids. Further, the insulator 108 may be an electrical or thermal insulator. It should be noted that, in some examples, the shield 106 and the insulator 108 may be provided by a single material, may be provided in one or more layers, may be provided by multiple materials and fabrics, and combinations thereof. The presently disclosed subject matter is not limited to the shield 106 and the insulator 108 as being single, separate layers, as FIG. 1 is merely an example for purposes of illustration.

The sensor 100 further includes a textile support 110. The textile support 110 may be a polymer, plastic, natural fiber, or combinations thereof, upon which an outer surface of the sensor 100 is affixed. The sensor 100 may be permanently or temporarily affixed to the textile support 110 using glue, adhesive, hook and loop fasteners, a snap fastener, a rubber band, and the like. The presently disclosed subject matter is not limited to any particular manner in which the sensor 100 is affixed to the textile support 110. The textile support 110 provides for the ability of the sensor 100 to be affixed to a garment, brief, gauze, or other fabric or material worn by a user of the sensor 100.

To affix the textile support 110 to a garment, brief, gauze, or other fabric or material worn by a user of the sensor 100, the textile support 110 may further include garment attachments 112A and 112B. The garment attachments 112A and 112B may be of various materials to allow the textile support 110 to be affixed, either permanently or temporarily, to the material worn by the user. The garment attachments 112A and 112B may include, but are not limited to, glue, adhesive, and/or hook and loop fasteners, snap fasteners, rubber band, and the like. The presently disclosed subject matter is not limited to any particular manner in which the textile support 110 is attached, either permanently or temporarily, to a material or the number or locations of the garment attachments 112A and 112B, as other methods or materials of attachment may be used and are considered to be within the scope of the presently disclosed subject matter. To apply a current or voltage, at various amperages, voltages, and/or frequencies, to measure capacitance, leads 114A and 114B are provided. As illustrated in FIG. 1, the lead 114A provides electrical power to the electrode 102A and the lead 114B provides electrical power to the electrode 102B. The leads 114A and 114B are attached to a monitoring system (not illustrated). The leads 114A and 114B may be constructed of various conductive or semiconductive material.

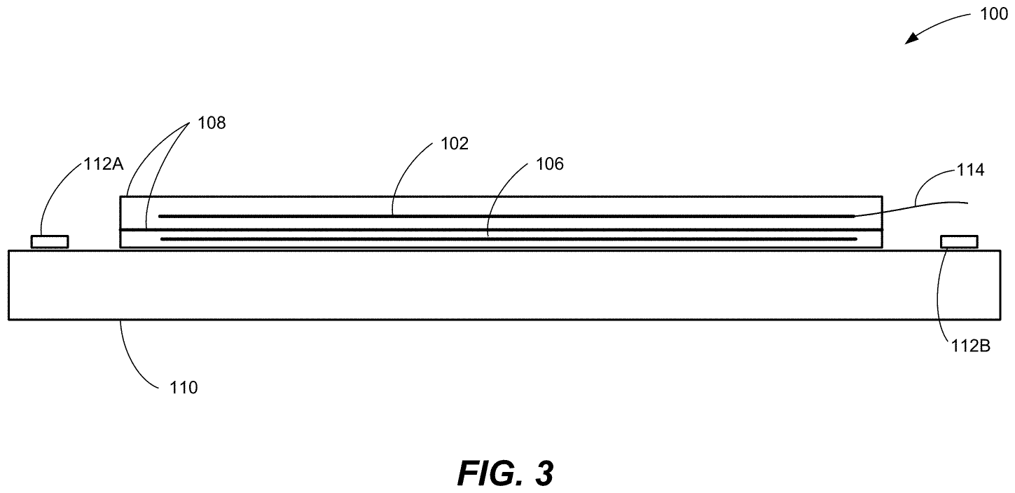

FIG. 3 is side, cutaway view of the sensor 100, in accordance with some examples of the present disclosure. Illustrated are the electrodes 102, the shield 106, the insulator 108, the textile support 110, the garment attachments 112A and 112B, and the leads 114. It should be noted that the relative size and/or thicknesses illustrated in FIGS. 1 and 3 are merely illustrative and are not intended to be limiting in any manner, as various examples of the presently disclosed subject matter may use materials having relative thicknesses that differ from those illustrated in FIGS. 1 and 3.

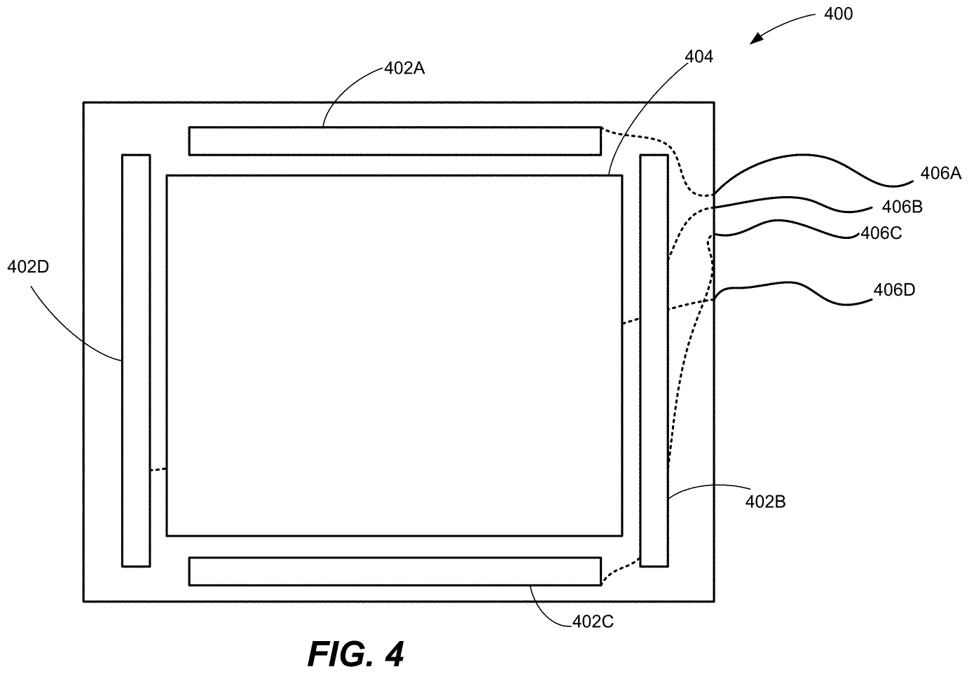

FIG. 4 illustrates a sensor apparatus 400, in accordance with some examples of the present disclosure. When detecting fluid, the use of a sensor, such as the sensor 100 of FIGS. 1 and 3, may be useful. However, the use of more other sensors may increase the accuracy and detectability of the presence or amount of fluid proximate to the sensors. The sensor apparatus 400 may be used to measure or detect one or more properties, such as electrical properties, of an absorbed fluid. In some examples, the additional sensor, such as the sensor apparatus 400 of FIG. 4, may be integrated with a capacitance sensor to form a single, combined unit (integrated solution). In another example, the additional sensor and the capacitance sensor may be two separate units (independent solution), as illustrated and described in FIG. 4. Thus, in FIG. 4, the sensor apparatus 400 may be used as part of an independent solution (as illustrated) or integrated into a capacitance sensor, such as one illustrated in FIGS. 1 and 2, as part of an integrated solution. As illustrated by way of example in FIG. 4, the number and spacing/layout of the electrodes 402A-402D may vary depending on the particular application, as the presently disclosed subject matter is not limited as such. In some examples, the electrodes 402A-402D may not be constructed nor operate in a manner similar to the sensor 100 of FIGS. 1 and 3. In some examples, the electrodes 402A-402D operate to produce an impedance reading, with two injection electrodes and two reading electrodes. It should be noted that the number of electrodes 402A-402D illustrated in FIG. 4 is merely an example, as more than four or fewer than four electrodes may be used and are considered to be within the scope of the presently disclosed subject matter. In some examples, the sensor apparatus 400 may include two electrodes, with one injection electrode and one reading electrode.

Within an inner space between the electrodes 402A-402D is a fluid region 404. The fluid region 404 is an area configured to receive at least a portion of a fluid. As noted above, the fluid may be urine, sweat, blood, diarrhea, or other bodily fluids. The fluid region 404 provides a space of definable area that may be used to generate a more accurate detection and measurement of bodily fluid. The fluid region 404 may be an absorbent material or adsorbent material, such as a clay or silica (gel) desiccant, or other types of materials such as sodium polyacrylate and other superabsorbent polymers such as, but not limited to, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. It should be noted that the presently disclosed subject matter is not limited to absorbent or adsorbent materials, and the use of one or the other in the following description is merely for purposes of illustration and not for limitation. The fluid region 404 may have the absorbent encased within a permeable membrane or material that allows the movement of the fluid into the absorbent. The electrodes of the sensors 402A-402D have leads 406A-406D connected to their respective electrodes. The leads 406A-406D provide an electrical connection between a monitoring or measuring system and the electrodes of the sensors 402A-402D.

When the absorbent in the fluid region absorbs fluid, the impedance of the electrodes 402A-402D begins to change. The reason for this is that the fluid affects the impedance between of the electrodes 402A-402D. A “dry” absorbent, i.e. one that has not absorbed a fluid, has different properties than a “wet” absorbent, i.e. one that has absorbed a fluid. These differing properties changes the dielectric properties that are measured by the sensor composed of electrodes 402A-402D. Thus, the impedance measured using the electrodes 402A-402D changes as the absorbent in the fluid region 404 absorbs fluid. Thus, if measured at a certain rate, the change in impedance may provide the ability to both detect the presence of an absorbed fluid as well as the rate of adsorption. The rate of adsorption may correlate to a flow rate (or volumetric flow rate) of the fluid.

For example, the sensor apparatus 400 may be affixed to an adult brief in an area likely to receive fluid should urine be expelled by a user while the adult brief is being word. A small droplet of urine may be absorbed by the absorbent in the fluid region 404. The electrodes 402A-402D may be used to detect a slight change in impedance when compared to a release of urine that saturates the absorbent in the fluid region 404. Further, the electrodes 402A-402D may be used to detect the range of change of the impedance as the absorbent goes from a “dry” condition to a saturated or at least wet condition, thus giving data that can be used to determine a rate of change (and thus flow rate) of the fluid.

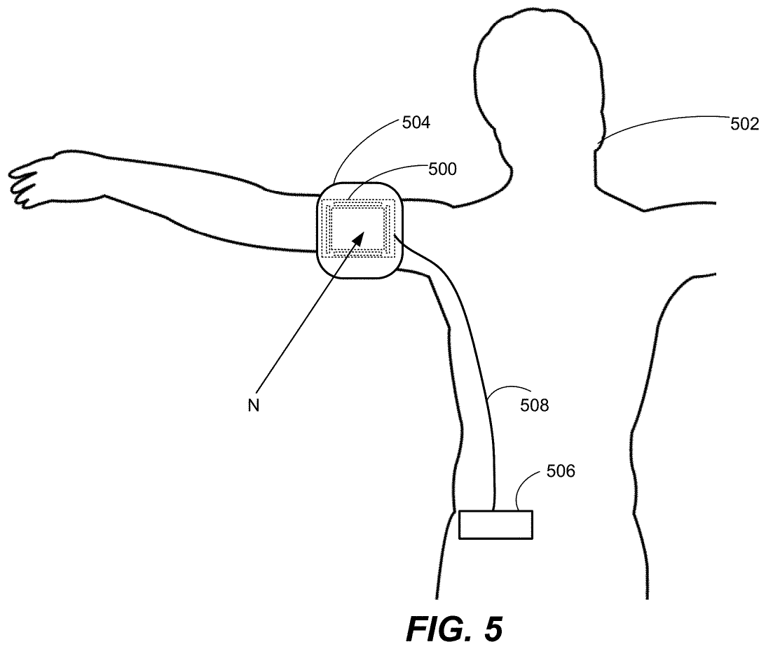

FIG. 5 is a front illustration of the placement of a sensor apparatus 500 for use in wound healing, in accordance with some examples of the present disclosure. The sensor apparatus 500 may be constructed and operate in a manner similar to the sensor apparatus 400 of FIG. 3, In some examples, the sensor apparatus 500 may be constructed and operate in a manner similar to other sensors, such as the sensors of FIGS. 1-3. In FIG. 3, a user 502 has a wound at location X that is wrapped in gauze 504. The sensor apparatus 500 is disposed on an inner surface of the gauze 504. In some examples, the sensor apparatus 500 may be placed within a layer of wrapping of the gauze 504. In other examples, the sensor apparatus 500 may be placed on the outside of the gauze 504 or proximate to the skin of the user 502. In further examples, there may be more than one sensor apparatus 500. In still further examples, the sensor apparatus 500 may have an absorbent selected for specific fluid.

As shown in FIG. 5, the sensor apparatus 500 is in electrical communication with a monitoring unit 506 through leads 508. The monitoring unit 506 may be configured to apply currents/voltages at various frequencies to measure the capacitance of the electrodes of the sensor apparatus 500. It should be understood that the use of the leads 508 as wires and the monitoring unit 506 as a separate component from the sensor apparatus 500 is merely for purposes of illustration and is not intended to be a limitation of the presently disclosed subject matter. For example, sensor apparatus 500 may include circuits or components that provide wireless transmission of the capacitance measurement (using a battery installed on the sensor apparatus to provide power) to the monitoring unit 506. In still further examples, the sensor apparatus 500 may have circuits or components that not only provide for the measurement of capacitance, but also the stored and output to an onboard (i.e. locally installed on the sensor apparatus 500) monitoring unit 506. Thus, in some examples, the various components described in FIG. 5 and the figures above may be one unit or may be multiple units.

In some examples, the monitoring unit 506 may output power at various frequencies to characterize the fluid(s) absorbed into the fluid region. Various fluids may cause the absorbent to have different dielectric changes. Thus, the use of different frequencies may not only be used to detect the type of fluid absorbed, but also the manner in which the fluid was absorbed (i.e. a fast discharge or slow discharge). In some examples, a timestamp may be generated to provide a beginning time and end time for discharge. A timestamp may be generated by detecting when the capacitance begins to change and when the capacitance change ends (or when the fluid is fully absorbed (saturated) into the absorbent material. In the example illustrated in FIG. 5, it may be useful to monitor for the discharge of blood from the wound on the arm. If blood is discharged, the fluid region of the sensor apparatus 500 may absorb at least a portion of the blood discharged. Using a change in capacitance as a correlation to flow rate, a person monitoring the user 502 may determine if the flow is significant or minor. Thus, the person monitoring the user 502 may be able to determine if the user 502 needs immediate medical attention or may be just experiencing a typical discharge found in normal wound healing. This may be used in other medical contexts. For example, a doctor monitoring a patient experiencing incontinence may be alerted that a significant discharge has occurred, and thereafter, be prompted to contact the patient to determine the condition(s) under which the discharge occurred.

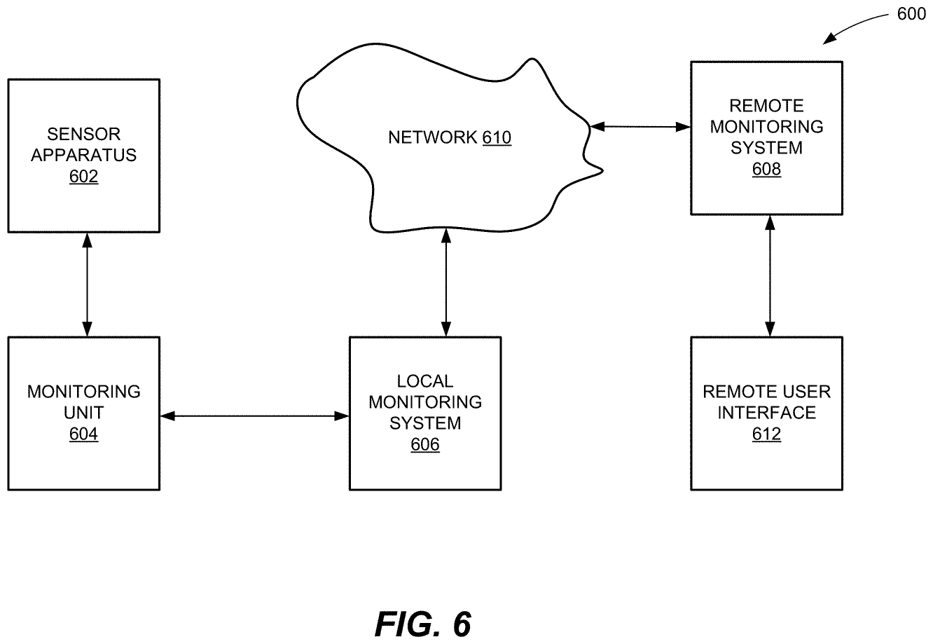

FIG. 6 is an illustration of a patient treatment system 600, in accordance with some examples of the present disclosure. It should be noted that the term “treatment” includes various monitoring, measuring, and diagnoses processes or procedures that may be used by personnel, such as a caretaker. Further, it should be noted that the illustration of individual components in FIG. 6 is merely an example, as the components may be combined with each other in various combinations. The presently disclosed subject matter is not limited to any particular physical or communicative configuration. The patient treatment system 600 may be used by medical care providers to monitor patients, such as the user 502 of FIG. 5. The patient treatment system includes a sensor apparatus 602, which may be constructed and operate in a manner similar to the sensor apparatus 400 of FIG. 4, the sensors described in FIGS. 1-4, or variations thereof. As described above, the sensor apparatus 602 uses a fluid region that absorbs various types of fluids discharged by a user. Electrodes within the sensor apparatus 602 are used to measure the capacitance change caused by the adsorption of the one or more fluids in the sensor apparatus 602. The patient treatment system 600 may use more than one sensor apparatus 602, the presently disclosed subject matter not being limited to one sensor apparatus 602. The sensor apparatus 602 may be installed on or within various garments, briefs. or other items worn by a user.

A monitoring unit 604, which may be constructed and operate in a manner similar to the monitoring unit 506 of FIG. 5, is in electrical communication with the sensor apparatus 602. The monitoring unit 604 provides the power to the sensor apparatus 602 and performs the capacitance measurement. The monitoring unit 604 is in communication with a local monitoring system 606. It should be noted that the communication pathways described in FIG. 6 are merely an example provided for purposes of illustration, as other communication pathways may be used. For example, the monitoring unit 604 may be in direct communication with the remote monitoring system through the network 610. For example, raw data may be transmitted directed from the monitoring unit 604 to the remote monitoring system 608 or even the remote user interface 612. The monitoring unit 604 may be in communication with various components, such as the local monitoring system 606, the remote monitoring system 608, and/or the remote user interface 612, through wired means, such as a physical cable, or wirelessly using Bluetooth (R) or other wireless technology. The local monitoring system 606 is used as the local control center for receiving instructions from a remote monitoring system 608 and for receiving data from the monitoring unit 604 for transmission to the remote monitoring system 608. The local monitoring system 606 may be various types of devices including, but not limited to, smart phones, mobile phones, cell phones, tablet computers, portable computers, laptop computers, personal digital assistants (PDAs), electronic book devices, or any other portable electronic devices that can generate, request, receive, transmit, or exchange voice, video, and/or digital data over a network.

The local monitoring system 606 is in communication with the remote monitoring system 608 through a network 610. The network 610 may be various types of networks, including, but not limited to, a cellular network or a wireless local network based on IEEE 802.11 standards. These and other type of networks are considered to be within the scope of the presently disclosed subject matter. The remote monitoring system 608 may be a web-based service accessible through an Internet interface or may be a service accessible through other secure connections.

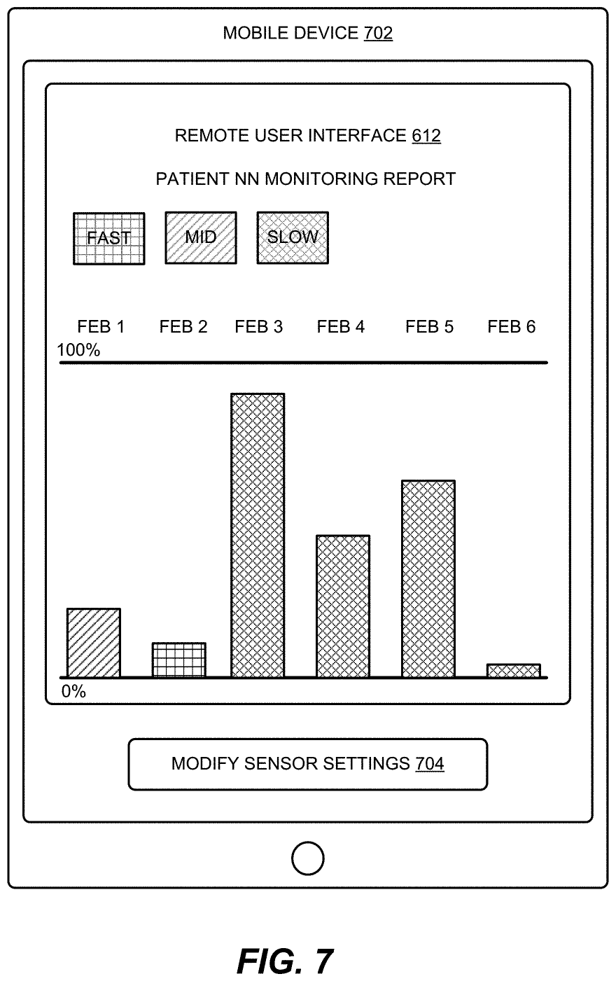

The remote monitoring system 608 stores data or facilitates the storage of data in a cloud based storage system and can also perform calculations. In some examples, the remote monitoring system 608 can receive data from the monitoring unit 604 and perform calculations such as, but not limited to, one or more fluid properties using the fluid properties sensor and a volume of fluid using the sensor apparatus. The remote monitoring system 608 further provides a communication link between the local monitoring system 606 and a remote user interface 612. In various examples, the remote user interface 612 may be a device used by a medical practitioner or caregiver that is monitoring or providing medical treatment to a user wearing the sensor apparatus 602. The remote user interface 612 may be configured to illustrate data transmitted from the local monitoring system 606. The remote user interface 612 may also be configured to perform other functions, such as accepting inputs from the caregiver to alter the functionality of the sensor apparatus 602 (such a changing power, frequency, and the like). The remote user interface 612 may be provided as an application on a device, as illustrated in FIG. 7.

FIG. 7 is an illustration of the remote user interface 612 as rendered on a mobile device 702, in accordance with some examples of the present disclosure. The mobile device 702 has rendered thereon the remote user interface 612. The remote user interface 612 has displayed thereon various information received from the local monitoring system 606. Illustrated in FIG. 7 are saturation readings of an amount of fluid absorbed by a sensor apparatus 602. It should be noted that the sensor apparatus 602 may be replaced by another sensor apparatus 602 for various reasons, including if the sensor apparatus 602 has absorbed a fluid or if the garment to which the sensor apparatus 602 is changed. Thus, the information rendered in the remote user interface 612 may be of more than one sensor apparatus 602.

In FIG. 7, the adsorption readings are provided for the days of February 1 through February 6. The adsorption readings for those days are rendered and graphed according to the percentage of adsorption from 0% to 100%. Further, the adsorption readings are rendered to show adsorption rates, from fast to slow. As illustrated, for example, the adsorption for the days of February 3-6 was slow, February 1 was mid, and February 2 was fast, meaning the sensor apparatus 602 detected various flowrates of the absorbed fluid on those days. The readings may have various degrees of granularity including, but not limited to, hourly readings and the like.

The remote user interface 612 may also include additional controls that allow the caregiver to modify the operation of the sensor apparatus 602. For example, the remote user interface 612 may include a modify sensor settings 704 interface. The modify sensor settings 704, when a selection is detected by the mobile device 702, may allow the caregiver to send instructions to the monitoring unit 604 to modify how the monitoring unit 604 operates, such as the frequency of voltage, power, current, detection times, and other settings. Further, the modify sensor settings 704 may further modify the output of the local monitoring system 606 to receive additional data. For example, the local monitoring system 606 may be a mobile communication device used and worn by the user. The caregiver, if wanting additional information, may request that the mobile communication device send information relating to the movement of the user as tracked by sensors located in the local monitoring system 606 (e.g. gyroscopes in cellular phones).

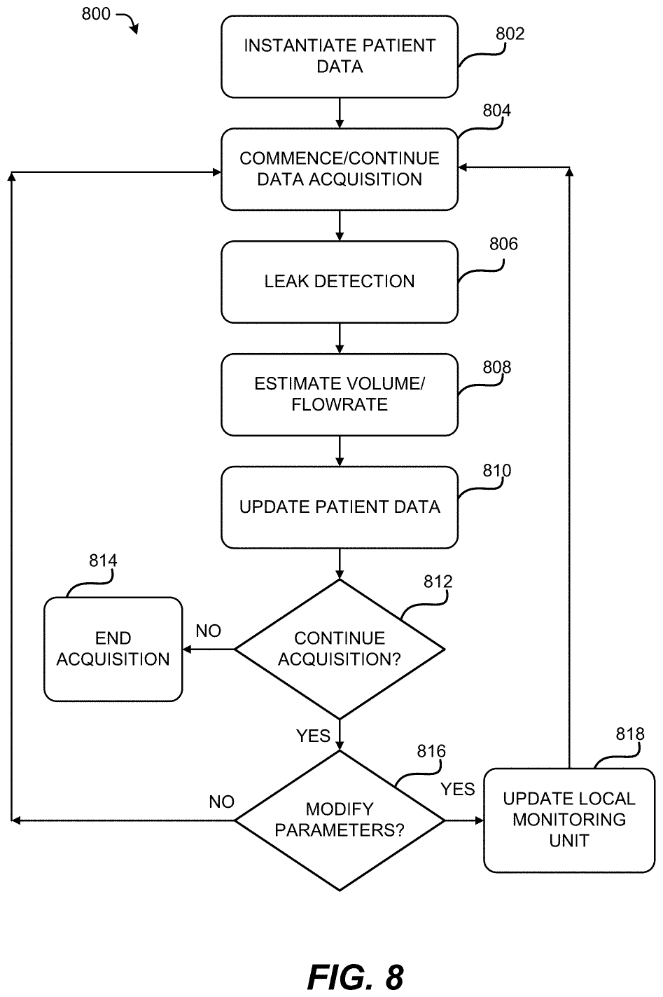

FIG. 8 is an example process 800 for fluid detection and quantification, in accordance with some examples of the present disclosure. The process 800 and other processes described herein are illustrated as example flow graphs, each operation of which may represent a sequence of operations that can be implemented in hardware, software, or a combination thereof. In the context of software, the operations represent computer-executable instructions stored on one or more tangible computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be omitted or combined in any order and/or in parallel to implement the processes.

The process 800 commences at operation 802, where patient data is instantiated. In some examples, the patent data includes information entered from a patient file, such as the patient identification and other health information. The patient data may also include the creation of a user profile for the patient so that a caregiver or medical practitioner can readily access the patient data using the user profile.

The process 800 continues to operation 804, where data acquisition commences. The commencement of data acquisition includes various operations. Using FIG. 6 as an example, the commencement of data acquisition includes establishing communications between the various systems and components, such as the monitoring unit 604, the local monitoring system 606, the remote monitoring system 608, and the remote user interface 612. The commencement of data acquisition also includes the commencement of the energizing of the electrodes of the sensor apparatus 602 to commence capacitance measurements. It should be noted that data acquisition at operation 804 continues while the process 800 continues to operation 806. The operation 804 may commence or stop for various reasons while the process continues from operation 804.

The process 800 continues to operation 806, where a leak, discharge, or presence of fluid is detected. The capacitance of the sensor apparatus 602 has changed to a degree indicating that a fluid has been absorbed by the sensor apparatus 602.

The process 800 continues to operation 808, where the volume and/or flowrate of the absorbed fluid is calculated. In some examples, the volume of the fluid may be determined using the change of capacitance. In further examples, the flowrate of the fluid may be determined using the rate of change of the capacitance of the sensor apparatus 602. It should be noted that other methods of estimating the volume and flowrate of fluids may be used and are considered to be within the scope of the presently disclosed subject matter.

The process 800 continues to operation 810, where the patient data from operation 802 is updated. The updating of patient data may be provided to a caregiver or medical practitioner on the remote user interface 612, whereby the view may see both current (i.e. real-time) and historical data.

The process 800 continues to operation 812, where a determination is made as to whether or not to continue acquisition of data from the sensor apparatus 602. If the determination is to cease acquisition, the process 800 ends at operation 814. If the determination is to continue acquisition, the sensor apparatus 602 may be changed to a new sensor apparatus 602 and the process continues to operation 816.

The process 800 continues to operation 816, where a determination is made as to whether or not parameters of the monitoring unit 604 (or other components) are to be modified. This may be done by various personnel, including caregivers and medical practitioners that may want to see additional or different data from the monitoring unit 604 (such as an increase of the frequency of the capacitance measurement).

If the determination at operation 816 is to not modify parameters, the sensor apparatus 602 may be changed to a new sensor apparatus 602 and the process may continue to operation 804. If the determination at operation 816 is to modify the parameters, the local monitoring unit 604 (for example) is updated, the sensor apparatus 602 may be changed to a new sensor apparatus 602 and the process may continue to operation 804.

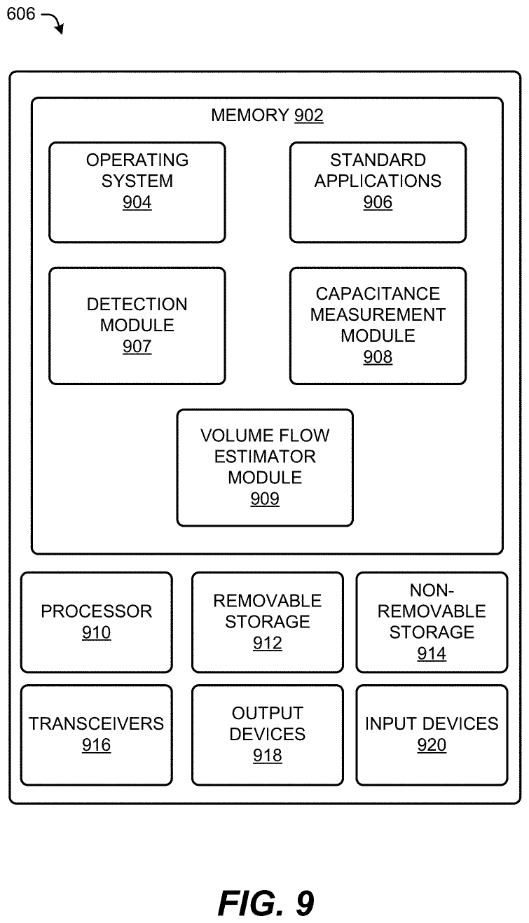

FIG. 9 depicts a component level view of the local monitoring system 606 of FIG. 6 for use with the systems and methods described herein. The local monitoring system 606 could be any device or combination of devices capable of providing the functionality associated with the systems and methods described herein. Although FIG. 6 describes the local monitoring system 606 as a physical layer, as noted above, various components, functions, modules, or other elements described herein may be provided by hardware, software, or combinations thereof. The local monitoring system 606 can comprise several components, modules, software functions, or computing devices to execute the above-mentioned functions. The local monitoring system 606 may be comprised of hardware, software, or various combinations thereof. The local monitoring system 606 may include functionality associated with other components, such as, but not limited to, the monitoring unit 604.

As discussed below, the local monitoring system 606 can comprise memory 902 including an operating system (OS) 904 and one or more standard applications 906. The standard applications 906 may include applications that provide for communication with the network 610. The OS 904 varies depending on the manufacturer of the local monitoring system 606. The OS 904 contains the modules and software that support basic functions of the local monitoring system 606, such as scheduling tasks, executing applications, and controlling peripherals. In some examples, the OS 904 can enable a detection module 907 that detects a change in capacitance of a sensor apparatus 602 that indicates the presence of a fluid, a capacitance measurement module 908 that receives data from the sensor apparatus 602 to determine a volume of a fluid, and a volume flow estimator module 909 that determines a flow rate of a fluid. The OS 904 can also enable the local monitoring system 606 to send and retrieve other data and perform other functions.

The local monitoring system 606 can also comprise one or more processors 910 and one or more of removable storage 912, non-removable storage 914, transceiver(s) 916, output device(s) 918, and input device(s) 920. In various implementations, the memory 902 can be volatile (such as random access memory (RAM)), non-volatile (such as read only memory (ROM), flash memory, etc.), or some combination of the two.

In some implementations, the processor(s) 910 can be one or more central processing units (CPUs), graphics processing units (GPUs), both CPU and GPU, or any other combinations and numbers of processing units. The local monitoring system 606 may also include additional data storage devices (removable and/or non-removable) such as, for example, magnetic disks, optical disks, or tape. Such additional storage is illustrated in FIG. 4 by removable storage 912 and non-removable storage 914. In some examples, data collected by a sensor, such as the sensors described in FIGS. 1-5 above, may be stored and processed at the local monitoring system 606. In other examples, data may be buffered in the local monitoring system 606 for processing by a remote unit, such as the remote monitoring system 608 or a cloud-based system that performs the processing functions described herein.

Non-transitory computer-readable media may include volatile and nonvolatile, removable and non-removable tangible, physical media implemented in technology for storage of information, such as computer readable instructions, data structures, program modules, or other data. The memory 902, removable storage 912, and non-removable storage 914 are all examples of non-transitory computer-readable media. Non-transitory computer-readable media include, but are not limited to, RAM, ROM, electronically erasable programmable ROM (EEPROM), flash memory or other memory technology, compact disc ROM (CD-ROM), digital versatile discs (DVD) or other optical storage, magnetic cassettes, magnetic tape, magnetic disk storage or other magnetic storage devices, or any other tangible, physical medium which can be used to store the desired information and which can be accessed by the local monitoring system 606. Any such non-transitory computer-readable media may be part of the local monitoring system 606 or may be a separate database, databank, remote server, or cloud-based server.

In some implementations, the transceiver(s) 916 include any transceivers known in the art. In some examples, the transceiver(s) 916 can include wireless modem(s) to facilitate wireless connectivity with other components (e.g., between the local monitoring system 606 and a wireless modem that is a gateway to the Internet), the Internet, and/or an intranet. Specifically, the transceiver(s) 416 can include one or more transceivers that can enable the local monitoring system 606 to send and receive data using the network 610. Thus, the transceiver(s) 916 can include multiple single-channel transceivers or a multi-frequency, multi-channel transceiver to enable the local monitoring system 606 to send and receive video calls, audio calls, messaging, etc. The transceiver(s) 916 can enable the local monitoring system 606 to connect to multiple networks including, but not limited to 2G, 3G, 4G, 5G, and Wi-Fi networks. The transceiver(s) can also include one or more transceivers to enable the local monitoring system 606 to connect to future (e.g., 6G) networks, Internet-of-Things (IoT), machine-to machine (M2M), and other current and future networks.

The transceiver(s) 916 may also include one or more radio transceivers that perform the function of transmitting and receiving radio frequency communications via an antenna (e.g., Wi-Fi or Bluetooth®). In other examples, the transceiver(s) 916 may include wired communication components, such as a wired modem or Ethernet port, for communicating via one or more wired networks. The transceiver(s) 916 can enable the local monitoring system 606 to facilitate audio and video calls, download files, access web applications, and provide other communications associated with the systems and methods, described above.

In some implementations, the output device(s) 918 include any output devices known in the art, such as a display (e.g., a liquid crystal or thin-film transistor (TFT) display), a touchscreen, speakers, a vibrating mechanism, or a tactile feedback mechanism. Thus, the output device(s) can include a screen or display. The output device(s) 918 can also include speakers, or similar devices, to play sounds or ringtones when an audio call or video call is received. Output device(s) 918 can also include ports for one or more peripheral devices, such as headphones, peripheral speakers, or a peripheral display.

In various implementations, input device(s) 920 include any input devices known in the art. For example, the input device(s) 920 may include a camera, a microphone, or a keyboard/keypad. The input device(s) 920 can include a touch-sensitive display or a keyboard to enable users to enter data and make requests and receive responses via web applications (e.g., in a web browser), make audio and video calls, and use the standard applications 906, among other things. A touch-sensitive display or keyboard/keypad may be a standard push button alphanumeric multi-key keyboard (such as a conventional QWERTY keyboard), virtual controls on a touchscreen, or one or more other types of keys or buttons, and may also include a joystick, wheel, and/or designated navigation buttons, or the like. A touch sensitive display can act as both an input device 920 and an output device 918.

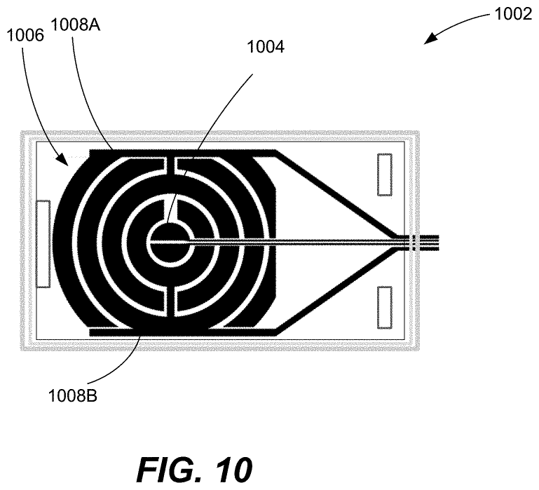

FIG. 10 is a top down illustration of a dual sensor 1002 providing for an integrated solution that may be used in conjunction with other examples described herein. In some examples, the use of a single sensor, such as the sensor 100 of FIG. 1, in an apparatus, such as the sensor apparatus 500 of FIG. 5, may not be as accurate or provide a desired amount of data. In these and other examples, the dual sensor 1002 may be used. The dual sensor 1002 may include both a fluid properties sensor 1004 and a capacitance sensor 1006. In some examples, the dual sensor 1002 is an integrated using having both the fluid properties sensor 1004 and the capacitance sensor 1006 in a single unit. The fluid properties sensor 1004 may operate in a manner similar to that described in FIG. 4. In other examples, the dual sensor 1002 may include two sensors, the fluid properties sensor 1004 and the capacitance sensor 1006, in different packages. The presently disclosed subject matter is not limited to any particular packaging of the fluid properties sensor 1004 and the capacitance sensor 1006.

The capacitance sensor 1006 may operate in a manner similar to the sensor 100 of FIG. 1, whereby electrodes 1008A and 1008B are used to measure capacitance. In some examples, the sensor 1006 is part of a resonance circuit, wherein the introduction of liquid proximate to the sensor 1006 changes the capacitance, thus changing the resonance frequency of the resonance circuit. This change in the resonance frequency is used to determine the capacitance and other data.

The fluid properties sensor 1004 may be used, among other functions, to provide a baseline or calibration of the capacitance sensor 1006. In some examples, properties of the fluid or fluids effecting the capacitance detected by the capacitance sensor 1006 may be needed or useful to determine qualitative or quantitative measurements. Further, because of the relative size of the capacitance sensor 1006, the effect of fluid absorbed proximate to the capacitance sensor 1006 may not provide a reliable indication of the liquid absorption. For example, fluid may be absorbed in one area of the capacitance sensor 1006 but not in another portion. In another example, the fluid absorbed may have difference properties (such as clarity, density, and the like).

In these and other examples, the fluid properties sensor 1004 may be used as means to calibrate or provide additional information. In some examples, the fluid properties sensor 1004 may be a permittivity sensor whereby the area proximate to the fluid properties sensor 1004 may configured to be saturated. In this example, the fluid properties sensor 1004 may be used to determine the permittivity of the fluid being absorbed. Because the permittivity is based on a saturated condition, the permittivity may be used to calculate the overall absorption occurring with respect to the capacitance sensor 1006, as the fluid properties sensor 1004 can provide the saturated baseline to which the measured capacitance of the capacitance sensor 1006 may be compared to. In this example, the capacitance sensor 1006 may be used to detect a volume of absorbed fluid and the fluid properties sensor 1004 may be used to detect a property of the fluid. The properties include, but are not limited to, electrical, optical, density, capacitance, impedance, temperature, and the like.

In another example, the fluid properties sensor 1004 may be an optical sensor that measures the clarity of the absorbed liquid. This may be useful in examples in which the type of fluid being absorbed may be needed. For example, in plastic surgery, a tight fitting garment is used to compress the body area after liposuction. It is not unusual, and desired, to allow liquid such as saline solution, to drain from the openings for the liposuction. However, in some examples, the area may become infected. In a case in which the liquid is infectious, the liquid will have different optical characteristics. Thus, the fluid properties sensor 1004 that uses an optical sensor may detect an infectious drainage rather than a typical saline drain. In these and other examples, the fluid properties sensors 1004 may be integrated into and communicate with a monitoring unit that controls data acquisition.

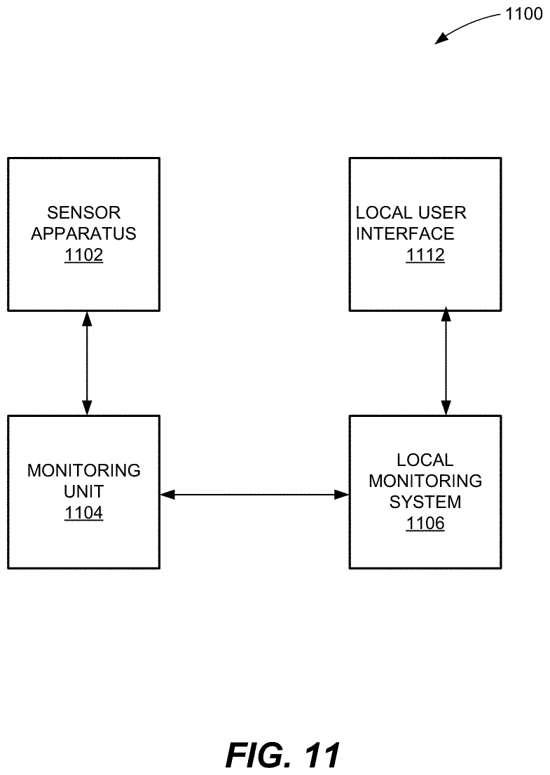

FIG. 11 is an illustration of a local patient treatment system 1100, in accordance with some examples of the present disclosure. It should be noted that the term “treatment” includes various monitoring, measuring, and diagnoses processes or procedures that may be used by personnel, such as a caretaker. Further, it should be noted that the illustration of individual components in FIG. 11 is merely an example, as the components may be combined with each other in various combinations. The presently disclosed subject matter is not limited to any particular physical or communicative configuration. The local patient treatment system 1100 may be used by medical care providers to monitor patients, such as the user 502 of FIG. 5. The local patient treatment system 1100 includes a sensor apparatus 1102, which may be constructed and operate in a manner similar to the sensor apparatus 400 of FIG. 4, the sensors described in FIGS. 1-4, or variations thereof. As described above, the sensor apparatus 1102 uses a fluid region that absorbs various types of fluids discharged by a user. Electrodes within the sensor apparatus 1102 are used to measure the capacitance change caused by the adsorption of the one or more fluids in the sensor apparatus 1102. The local patient treatment system 1100 may use more than one sensor apparatus 1102, the presently disclosed subject matter not being limited to one sensor apparatus 1102. The sensor apparatus 1102 may be installed on or within various garments, briefs. or other items worn by a user.

A monitoring unit 1104, which may be constructed and operate in a manner similar to the monitoring unit 506 of FIG. 5, is in electrical or wireless communication with the sensor apparatus 1102. The monitoring unit 1104 can provide the power to the sensor apparatus 1102 if no local power (such as a battery) is used, as well as performs the capacitance measurement. The monitoring unit 1104 is in communication with a local monitoring system 1106. The local monitoring system 1106 may be various types of devices including, but not limited to, smart phones, mobile phones, cell phones, tablet computers, portable computers, laptop computers, personal digital assistants (PDAs), electronic book devices, or any other portable electronic devices that can generate, request, receive, transmit, or exchange voice, video, and/or digital data over a network.

The local monitoring system 1106 is in communication with the local user interface 1112. The local user interface 1112 may be various types of devices including, but not limited to, smart phones, mobile phones, cell phones, tablet computers, portable computers, laptop computers, personal digital assistants (PDAs), electronic book devices, or any other portable electronic devices that can generate, request, receive, transmit, or exchange voice, video, and/or digital data.

The local monitoring system 1106 or other components, such as the local user interface 1112 may store data or facilitates the storage of data locally or in a cloud based storage system and can also perform calculations. In some examples, the local monitoring system 1106 can receive data from the monitoring unit 1104 and perform calculations such as, but not limited to, one or more fluid properties using the fluid properties sensor and a volume of fluid using the sensor apparatus. In various examples, the local user interface 1112 may be a device used by a medical practitioner, a user, or caregiver that is monitoring or providing medical treatment to a user wearing the sensor apparatus 1102. The local user interface 1112 may be configured to illustrate data in a display transmitted from the local monitoring system 1106. The local user interface 1112 may also be configured to perform other functions, such as accepting inputs from the caregiver to alter the functionality of the sensor apparatus 1102 (such a changing power, frequency, and the like). The local user interface 1112 may be provided as an application on a device, as illustrated in FIG. 7. The local monitoring system 1106, or other components such as the local user interface 1112, can provide various notifications upon a determination of a result of various calculations, such as fluid properties or volume. The notifications may include, but are not limited to, alarms, messages, and the like.

The presently disclosed examples are considered in all respects to be illustrative and not restrictive. The scope of the disclosure is indicated by the appended claims, rather than the foregoing description, and all changes that come within the meaning and range of equivalents thereof are intended to be embraced therein.

What is claimed is:

1. A system comprising:
a memory storing computer-executable instructions; and
a processor in communication with the memory, the computer-executable instructions causing the processor to perform acts comprising:
receiving an instruction to commence monitoring for a fluid using a sensor apparatus, wherein the sensor apparatus comprises a capacitance sensor and a fluid properties sensor;
instructing a monitoring unit to commence data acquisition by energizing the sensor apparatus to determine a capacitance of the sensor apparatus using the capacitance sensor;
receiving a plurality of data from the monitoring unit at periodic intervals; and
transmitting the plurality of data to a remote monitoring system to determine, at the remote monitoring system, one or more fluid properties using the data of the fluid properties sensor, and fluid volume using capacitance sensor data and at least one of fluid properties.

2. The system of claim 1, further comprising computer-executable instructions that cause the
processor to perform acts comprising:
receiving from the remote monitoring system at least one of:
one or more fluid properties using the fluid properties sensor data; and
a volume of fluid absorbed by the sensor apparatus for at least one of the periodic intervals using at least one of the plurality of data.

3. The system of claim 1, wherein the sensor apparatus is continuously energized.

4. The system of claim 1, wherein the sensor apparatus is energized at periodic intervals.

5. The system of claim 1, wherein fluid properties data is determined after the capacitance.

6. The system of claim 1, further comprising computer-executable instructions that cause the processor to perform acts comprising determining a timestamp indicating a beginning of fluid discharge and an end of fluid discharge.

7. The system of claim 1, wherein the fluid properties sensor comprises a permittivity sensor.

8. The system of claim 1, wherein the fluid properties sensor comprises an optical sensor.

9. The system of claim 1, wherein the fluid properties sensor comprises a second capacitance sensor having a size smaller than the capacitance sensor.

10. The system of claim 1, wherein the fluid properties sensor comprises a temperature sensor.

11. The system of claim 1, wherein the fluid properties sensor is an impedance sensor.

12. The system of claim 1, wherein the one or more fluid properties is measured at various intervals.

13. The system of claim 1, wherein the instruction to commence monitoring is generated by a remote user interface.

14. The system of claim 1, wherein the fluid comprises urine, sweat, blood, diarrhea, or other bodily fluids.

15. The system of claim 1, wherein the monitoring is for one of spontaneous cerebrospinal fluid leak, amniotic fluid leakage, lymphorrhagia, exuding wounds, diarrhea, vomiting, excessive tear secretion for a person in a coma, excessive secretion of saliva for a person in a coma, incontinence, and excessive sweating at night a sign of low blood sugar.

16. The system of claim 1, wherein the monitoring is for one of fibroids, endometriosis, adenomyosis, pelvic inflammatory disease (PID), endometrial polyps, cancer of a womb, polycystic ovary syndrome (PCOS), blood clotting disorders such as Von Willebrand disease, an underactive thyroid gland (hypothyroidism), and diabetes.

17. The system of claim 1, wherein the sensor apparatus comprises a fluid region comprising an absorbent to absorb at least a portion of the fluid, wherein the absorbent comprises clay desiccant, silica (gel) desiccant, sodium polyacrylate, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

18. The system of claim 1, wherein the capacitance sensor comprises:
two electrodes;
a shield; and
an insulator encapsulating the two electrodes and the shield.

19. The system of claim 18, wherein the two electrodes are shaped to have mirror image sides to reduce an effect of deformation of the sensor.

20. The system of claim 1, wherein the sensor apparatus is affixed to a garment, fabric, or brief worn by a user being monitored.

21. The system of claim 1, further comprising computer-executable instructions that cause the processor to perform acts comprising modifying the periodic intervals.

22. The system of claim 1, further comprising computer-executable instructions that cause the processor to perform acts comprising storing the plurality of data for access by a caregiver or medical practitioner.

23. The system of claim 1, further comprising computer-executable instructions that cause the processor to perform acts comprising determining a volumetric flowrate of the fluid.

24. The system of claim 23, wherein determining the volumetric flowrate is based on a rate of change in a capacitance of the sensor apparatus.

25. A method of treating a patient by monitoring for a presence of a fluid, the method comprising:
instructing the patient to wear a garment having disposed therein a sensor apparatus and a monitoring unit, the sensor apparatus positioned proximate to an area of a body of the patient desired for monitoring, wherein the sensor apparatus comprises a capacitance sensor for determining a capacitance and a fluid properties sensor for determining at least one fluid property;
instructing a local monitoring system to instruct the monitoring unit to commence data acquisition comprising measurements of the capacitance using the capacitance sensor and the at least one fluid property using the fluid properties sensor at periodic intervals;
receiving determinations of a volume or a volumetric flowrate of an absorbed fluid using the capacitance determined using the capacitance sensor and the one or more fluid properties using the fluid properties sensor; and
analyzing the determinations of the volume or the volumetric flowrate of the fluid and determine a treatment based on the determinations of the volume or the volumetric flowrate of the fluid.

26. The method of claim 25, further comprising transmitting an instruction to the local monitoring system to modify at least one parameter of the sensor apparatus based on the treatment determined using the determinations of the volume or the volumetric flowrate of the fluid.

27. The method of claim 25, wherein the fluid comprises sweat, blood, diarrhea, urine, or other bodily fluids.

28. The method of claim 25, wherein the monitoring is for one of spontaneous cerebrospinal fluid leak, amniotic fluid leakage, lymphorrhagia, exuding wounds, diarrhea, vomiting, excessive tear secretion for a person in a coma, excessive secretion of saliva for a person in a coma, excessive sweating at night a sign of low blood sugar, fibroids, endometriosis, adenomyosis, pelvic inflammatory disease (PID), endometrial polyps, cancer of a womb, polycystic ovary syndrome (PCOS), blood clotting disorders such as Von Willebrand disease, an underactive thyroid gland (hypothyroidism), stress (urinary) incontinence, urgency (urinary) incontinence, postural (urinary) incontinence, mixed (urinary) incontinence, and incontinence associated with chronic retention of urine, and diabetes.

29. The method of claim 25, wherein the sensor apparatus comprises a plurality of sensors and a fluid region, the fluid region comprising an absorbent to absorb at least a portion of the fluid, wherein each of the plurality of sensors comprise:
at least two electrodes;
a shield; and
an insulator encapsulating the two electrodes and the shield.

30. The method of claim 29, wherein the at least two electrodes are shaped to have mirror image sides to reduce an effect of deformation of the sensor.

31. The method of claim 25, wherein the sensor apparatus is affixed to a garment, fabric, or brief worn by the patient.

32. The method of claim 25, further comprising modifying the periodic intervals.

33. A sensor apparatus for fluid monitoring, the sensor apparatus comprising:
at least two capacitive sensing electrodes shaped to have at least one mirror image axes of deformation to reduce an effect on measurement of deformation of the sensor;
a shield;
a fluid region comprising absorbent encapsulated within a permeable membrane, the fluid region configured for absorbing at least a portion of the fluid;
an insulator encapsulating the two capacitive sensing electrodes and the shield; and
a textile support.

34. The sensor apparatus of claim 33, further comprising a second shield covering at least a portion of the two capacitive sensing electrodes.

35. The sensor apparatus of claim 33, wherein the absorbent comprises clay desiccant, silica (gel) desiccant, sodium polyacrylate, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

36. The sensor apparatus of claim 33, further comprising at least one garment attachment on the textile support for attaching the sensor apparatus to a garment, brief, or material worn by a user being monitored.

37. The sensor apparatus of claim 33, further comprising a plurality of leads, each of the plurality of leads in electrical communication with the at least two capacitive sensing electrodes.

38. The sensor apparatus of claim 37, wherein the plurality of leads are in electrical communication with a monitoring unit, whereby the monitoring unit energizes at least two capacitive sensing electrodes through the plurality of leads to measure a capacitance of the sensor apparatus.

39. A system comprising:
a memory storing computer-executable instructions; and
a processor in communication with the memory, the computer-executable instructions causing the processor to perform acts comprising:
receiving an instruction to commence monitoring for a fluid using a sensor apparatus, wherein the sensor apparatus comprises a capacitance sensor and a fluid properties sensor;
instructing a monitoring unit to commence data acquisition by energizing the sensor apparatus to determine a capacitance of the sensor apparatus using the capacitance sensor and to determine one or more fluid properties using the fluid properties sensor;
receiving a plurality of data from the monitoring unit at periodic intervals;
determining a result of at least one of:
one or more fluid properties using the fluid properties sensor; and
a volume of fluid absorbed by the sensor apparatus for at least one of the periodic intervals using at least one of the plurality of data; and
upon determining the result, providing a notification of an alarm rise or a message on a display.

40. The system of claim 39, further comprising computer-executable instructions that cause the processor to perform the act comprising storing the result.

41. The system of claim 39, further comprising computer-executable instructions causing the processor to perform acts comprising transmitting the at least one of the one or more fluid properties or the volume of fluid to a remote monitoring system.
